(19) United States
(12) Patent Application Publication
(10) Pub. No.: US 2009/0246149 A1
(43) Pub. Date: Oct. 1, 2009

(54) MEDICINAL AEROSOL FORMULATIONS

(75) Inventors: Philip A. Jinks, Leicestershire (GB); Lesley McKenzie, Glasgow (GB); James T. Lister, Nottinghamshire (GB)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 12/472,088

(22) Filed: May 26, 2009

Related U.S. Application Data
(63) Continuation of application No. 10/398,335, filed on Oct. 6, 2003, now abandoned.

(30) Foreign Application Priority Data
Oct. 9, 2000 (GB) 0024711.4
Sep. 18, 2001 (GB) 0122512.7

(51) Int. Cl. A61K 9/12; A61P 11/00

(52) U.S. Cl. 424/45; 424/43

(57) ABSTRACT

Use of particulate bulking agents having an extremely small mass median diameter of less than one micron, preferably less than 300 nm, in pharmaceutical aerosol formulations comprising a suspension of drug particles in a propellant. Examples of bulking agents include ascorbic acid, saccharides, polysaccharides, amino acids, organic and inorganic salts, urea, and propyliodone.

### FIELD OF THE INVENTION

This invention relates to medicinal aerosol formulations and in particular to suspension aerosol formulations containing drug particles and a nanoparticulate auxiliary powder suitable for administration to the respiratory tract.

### BACKGROUND

Medicinal aerosol formulations in pressurised containers have been available for over forty years. For most of this time, chlorofluorocarbons have been used as the propellants. Drugs have been formulated either as solutions or as suspensions, depending on their solubility properties and other factors. Following environmental concerns over their use, other propellants have been introduced, and this has presented a challenge to reformulate or to introduce new drugs, as well as an opportunity to provide improved pharmaceutical performance.

Two propellants that have emerged as favourites are 1,1,1,2-tetrafluoroethane (HFA 134a) and 1,1,1,2,3,3,3-heptafluoropropane (HFA 227). These have distinctly different solvent properties to the chlorofluorocarbons, and this has had a bearing on the properties of formulations.

When formulating suspensions, micronised drug is dispersed in a propellant system with other ingredients added as appropriate for maintaining the stability of the formulation. One aspect of stability is the homogeneity of the dispersed drug, which can sediment (settle) or cream (float) depending on the density difference between drug and propellant, or it can flocculate, which requires some degree of agitation to deflocculate it. Such challenges are presented when formulating suspensions of any drug, but are particularly important when high potency drugs, such as Formoterol, Fluticasone Propionate, Salmeterol, Procaterol and Ipratropium and salts thereof are formulated.

When more potent drugs are formulated as suspensions, the concentration of drug required is lower than for less potent drugs. Sedimenting, creaming or flocculating drug leads to greater inhomogeneity of the contents that in turn may lead to delivery of incorrect doses when the formulation is dispensed from the metering valve.

Formulations of more potent drugs have been prepared as suspensions in hydrofluoroalkanes, such as those disclosed in WO 97/47286 (herein incorporated by reference) but it is still possible to make further improvements to the homogeneity of such formulations.

Various suggestions have been made to improve the quality of pharmaceutical aerosol formulations.

WO 00/27363 and U.S. Pat. No. 5,747,001 disclose aerosol formulations comprising droplets of an aqueous dispersion of nanoparticles comprising insoluble drug having a surface modifier on the surface thereof. The nanoparticles generally have an effective average particle size of less than about 1000 nm.

EP-A-0768114 discloses a method for homogenizing and micronising aerosol formulations in a closed apparatus under elevated pressure. The apparatus includes a closed loop containing a reaction vessel and homogenizer. The homogenizer includes an interaction chamber and an intensifier pump; the interaction chamber includes a stream splitter for separating a stream of aerosol formulation into two streams and an impaction chamber for combining the streams. Aerosol formulations comprising ipratropium bromide and albuterol sulphate and optionally surfactant in CFC propellants and HFA 227 are disclosed with particle sizes of 5 to 10 micron.

U.S. Pat. No. 5,711,934 discloses a process for preparing aerosol formulations by milling the formulation in aerosol propellant at a temperature between −80° C. and 10° C. Particle sizes of less than 10 micron are obtained.

EP-A-0726088 discloses a process for preparing aerosol compositions in which the composition is maintained under constant recirculation under high pressure conditions and forced through plates with a plurality of micro-apertures until obtaining a uniform dispersion. No particle sizes are disclosed.

WO 00/25746 disclose a process for the preparation of suspensions of drug particles for inhalation delivery which includes the step of homogenizing the formulation in a turboemulsifier provided with a high-potency turbine, optionally followed by a treatment in a high-pressure homogenizer. The Examples demonstrate the preparation of an aqueous suspension of beclomethasone dipropionate.

U.S. Pat. No. 6,086,376 discloses aerosol formulations containing stabilized particles of drug having a mean size range of 0.1 to 10 micron coated with a membrane-forming, amphiphatic lipid and dispersed in HFA 134a or HFA 227 propellant.

U.S. Pat. No. 5,858,410 discloses a drug carrier comprising particles of at least one pure active compound which is insoluble, only sparingly soluble or moderately soluble in water, aqueous media and/or organic solvents, wherein the active ingredient is solid at room temperature and has an average diameter, determined by photon correlation spectroscopy of 10 nm to 1000 nm, the proportion of particles larger than 5 micron in the total population being less than 0.1% (number distribution). It is suggested that for metered aerosols spraying a powder the drug carriers in the nanometre range are sprayed onto carrier particles, such as lactose, in the micrometre range. The lactose dissolves in the lung releasing the drug carriers.

### SUMMARY OF THE INVENTION

In one aspect the present invention relates to the use of a particulate bulking agent having a mass median diameter of less than one micron in pharmaceutical aerosol formulations comprising drug in a suspension of particles in a propellant.

In accordance with a second aspect of the invention there is provided a pharmaceutical aerosol formulation comprising particles of drug dispersed in a propellant and a bulking agent having a mass median diameter of less than one micron.

### DETAILED DESCRIPTION

It has been found that improved suspension aerosol formulations of drug can be prepared by introducing a bulking agent having a mass median diameter of less than one micron. The bulking agent improves the stability of the suspension of drug particles which may be micronised drug particles or other drug particles having a mass median diameter equal to or greater than 1 micron (more particularly from 1 to 10 micron, even more particularly from 1 to 5 micron) or smaller particles having a mass median diameter of less than one micron. It is not necessary for the surface of the bulking agent or the drug to be coated with a surface modifier to achieve improved stability.

Mass median diameter (which is equivalent to volume median diameter) can be determined using any conventional particle size measurement method known to those skilled in the art. Suitable methods include for example laser diffraction, photon correlation spectroscopy (e.g. using a spectrometer available under the trade designation Brookhaven PCS from Brookhaven Inc.), spinning disc centrifuging (using an instrument available under the trade designation CPS Disc Centrifuge from Chemical Process Specialists Inc.), and scanning electron microscopy (SEM). Mass median diameter is preferably determined by laser diffraction, more particularly laser diffraction using an analyser available under the trade designation Malvern Mastersizer 2000 laser light diffraction particle size analyser from Malvern Instruments Ltd.

Although potent drugs (i.e. a drug having a potency such that concentrations of the drug in a formulation of less than about 0.1% w/w are therapeutically effective), such as Formoterol, Fluticasone Propionate, Salmeterol, Procaterol, Ipratropium and salts thereof benefit particularly from this invention, it is applicable to any medicament to be prepared as a suspension formulation. Other drugs which may be used in aerosol formulations are well known and are referred to in the above literature references. Non-limiting examples of other suitable drugs include antiallergics, analgesics, bronchodilators, antihistamines, therapeutic proteins and peptides, antitussives, anginal preparations, antibiotics, anti-inflammatory preparations, hormones, or sulfonamides, such as, for example, a vasoconstrictive amine, an enzyme, an alkaloid or a steroid, and combinations of these specific examples or drugs which may be employed are: isoproterenol [alpha-(isopropylaminomethyl) protocatechuyl alcohol], phenylephrine, phenylpropanolamine, glucagon, adrenochrome, trypsin, epinephrine, ephedrine, narcotine, codeine, atropine, heparin, morphine, dihydromorphinone, ergotamine, scopolamine, methapyrilene, cyanocobalamin, terbutaline, rimiterol, salbutamol, isoprenaline, fenoterol, oxitropium bromide, reproterol, budesonide, flunisolide, ciclesonide, triamcinolone acetonide, mometasone furoate, colchicine, pirbuterol, beclomethasone dipropionate, orciprenaline, fentanyl, and diamorphine. Others are antibiotics, such as neomycin, streptomycin, penicillin, procaine penicillin, tetracycline, chlorotetracycline and hydroxytetracycline; adrenocorticotropic hormone and adrenocortical hormones, such as cortisone, hydrocortisone, hydrocortisone acetate and prednisolone; antiallergy compounds such as cromolyn sodium, nedocromil, protein and peptide molecules such as insulin, pentamidine, calcitonin, amiloride, interferon, LHRH analogues, DNAase, heparin, etc. The drugs exemplified above may be used as either the free base or as one or more salts known to the art. Vaccines may also benefit from this approach.

Preferred bulking agents include lactose, DL-alanine, ascorbic acid, glucose, sucrose D(+)trehalose as well as their various hydrates, anomers and/or enantiomers. Lactose (including its various forms, such as α-lactose monohydrate and β-lactose) is more preferred as a bulking agent due to e.g. processing considerations. Other suitable bulking agents include other saccharides e.g. D-galactose, maltose, D(+)raffinose pentahydrate, sodium saccharin, polysaccharides e.g. starches, modified celluloses, dextrins or dextrans, other amino acids e.g. glycine, salts e.g. sodium chloride, calcium carbonate, sodium tartrate, calcium lactate, or other organic compounds e.g. urea or propyliodone.

The weight ratio of drug to bulking agent is generally in the range 1:0.1 to 1:100. Preferably the weight ratio is in the range 1:5 to 1:40, although lower ratios of drug to bulking agent will be required for less potent drugs.

The concentration of drug depends largely on its potency. This invention is particularly applicable to drugs formulated at a concentration of less than 0.1% w/w.

For drugs formulated at a concentration of less than 0.1% w/w, a weight ratio of drug to bulking agent in the range 1:10 to 1:30 has been found particularly suitable, and a weight ratio of about 1:20 most suitable. For drugs formulated at a concentration equal to or greater than 0.1% w/w, a weight ratio of drug to bulking agent in the range 1:0.1 to 1:10 has been found particularly suitable, a weight ratio in the range 1:0.5 to 1:5 even more suitable, and a weight ratio in the range 1:1 to 1:2 most suitable.

In other preferred embodiments according to the present invention, bulking agents include an active ingredient. In particular for pharmaceutical aerosol formulations comprising two (or more) drugs, one drug, besides acting as an active ingredient, can be desirably applied in the formulation as a particulate bulking agent in accordance with the present invention. Although the concentration of each drug in such a formulation depends largely on its potency, the weight ratios of drug to bulking agent as described above are applicable, wherein the drug applied as the bulking agent is understood under “bulking agent” and the other drug (or drugs) being bulked is understood under “drug”. This approach is particularly desirable for improving stability of suspension formulations wherein the drug (or drugs) being bulked has a mass median diameter equal to or greater than 1 micron (more preferably from 1 to 10 micron, most preferably from 1 to 5 micron). This approach is also particularly advantageous for pharmaceutical aerosol formulations comprising a potent drug and a second, preferably a less potent, drug, wherein the second drug is applied as a particulate bulking agent in the formulation. Preferred drug combinations include: a potent bronchodilator, such as Formoterol, Salmeterol, Procaterol, Ipratropium bromide and salts thereof, in combination with an anti-inflammatory, such as budesonide, flunisolide, ciclesonide, triamcinolone acetonide, mometasone furoate and beclomethasone dipropionate, as the bulking agent; and a potent anti-inflammatory, such as Fluticasone Propionate, in combination with a bronchodilator, such as isoproterenol, terbutaline, rimiterol, salbutamol, reproterol, pirbuterol, orciprenaline and salts thereof, as the bulking agent.

The bulking agent may be reduced to the required particle size by any convenient method, e.g. grinding, air-jet milling etc. Preferably the bulking agent is reduced to nanoparticle size in a high pressure homogenizer, such as the commercially available Avestin Emulsiflex homogenizers and the Microfluidics Microfluidizer homogenizers. Surprisingly in the processing with high pressure homogenizers, certain bulking agents can be reduced to the desired particle size using lower pressures than that applied for other bulking agents. For example, it has been found that lactose, more specifically

α-lactose monohydrate, can be effectively reduced to the desired particle size using pressures between about 10,000 and about 21,000 psi, while for effective particle size reduction of alanine or sucrose higher pressures of about 25,000 psi were applied. The mass median diameter of the bulking agent can advantageously be as low as 300 nanometers, more desirably as low as 250 nanometers and most desirably the mass median diameter is in the range of 100 to 250 nanometers.

The bulking agent may be prepared in a slurrying aid which is a low volatility solvent such as ethanol. It may be prepared in a slurrying aid which is a component of the final aerosol formulation, or it may be prepared in a solvent that is subsequently removed or exchanged with a component of the formulation by some process such as centrifugation and decanting, dialysis, evaporation etc. Volatile ingredients of the formulation may be used as the slurrying aid, such as propellants, provided the homogenizer and any associated pipework or product reservoirs are built to withstand the propellant pressure.

It is particularly convenient to use a slurrying aid in the high pressure homogenizer which is a low volatility component of the aerosol formulation and after particle size reduction has been achieved the slurry may be adjusted if necessary, e.g. concentrated by centrifugation, decanting etc. Whilst it has been found that slurries with excessively high powder loadings may be difficult to process due to their rheological properties, it is generally advantageous to process slurries with powder loading concentrations which approach this processing limit in order to achieve the desired particle size distribution in the shortest processing time. Thus, the weight ratio of liquid:solid is generally in the range 5:1 to 100:1, preferably 5:1 to 20:1, and most preferably about 10:1.

According to a further aspect of the invention there is provided a process for the preparation of an aerosol formulation comprising a suspension of particles in propellant which process comprises forming a slurry of bulking agent with a component of the aerosol formulation, subjecting the slurry to high pressure homogenization to reduce the particle size of the bulking agent, and thereafter mixing the resulting slurry with other components of the aerosol formulation.

The aerosol formulations of the invention may contain ethanol, generally in an amount in the range 0.1 to 5% by weight, preferably from about 0.5 to about 3% by weight, more preferably from about 1 to about 2% by weight.

The aerosol formulations of the invention may optionally contain surfactant. Suitable surfactants are well known in the art and include sorbitan trioleate, oleic acid and lecithin. Surfactants, such as oligolactic acid derivatives disclosed in WO94/21228 and WO94121229, and other surfactants disclosed in the literature may be used.

The invention will be illustrated by the following Examples.

### Example 1

α-lactose monohydrate supplied under the trade designation Pharmatose 325M by DMV International Pharma was micronised by fluid energy milling in a single pass (referred to here and in the following as “micronised lactose monohydrate”). Micronised lactose monohydrate (15 g) was then dispersed in anhydrous ethanol (500 g). This dispersion was added to the product reservoir of an Avestin Emulsiflex C50 homogeniser. The pressure was stepped up according to the following protocol:

| Length of time (mins) | Pressure (psi) |
|---|---|
| 5 | 0 |
| 5 | 5,000 |
| 5 | 10,000 |
| 10 | 15,000 |
| 5 | 15,000 |
| 10 | 18,000 |

Microscopic analysis of the product after 40 minutes showed it to be considerably less than 1 micron. The product was also examined by Scanning Electron Microscopy (SEM), and the particle size estimated to be 100 nm. Quantities of dispersions sampled after 5 minutes and after 40 minutes of the above protocol (1.5 g) were added to a 20 ml clear pressure resisting plastic vials and non-metering valves then crimped in place. HFA 134a (20 g) was then injected through each valve. The suspension formulation from the 40 minute sample was seen to have a markedly lower sedimentation rate and a larger sediment bulk on standing compared to the 5 minute sample, both features of which are advantageous for a bulking agent in metered dose inhaler suspension formulations. Hence a technique has been found to provide a stable suspension of an excipient (lactose) which in the particle size range typically used for inhalation (2 to 5 microns) would have too rapid a sedimentation rate to be adequately stable but when further reduced in size to 300 nanometres provides a stable dispersion. The technique is likely to be applicable to a wide range of other compounds which by the degree of their density difference in comparison with HFA propellants, would in the size range typically used in inhaler suspension formulations, form poorly stable dispersions.

### Example 2

A slurry of micronised lactose monohydrate (15 g) in anhydrous ethanol (500 g) was processed according to Example 1

The slurry of lactose was centrifuged for 5 minutes at 5000 rpm using 10 g per tube, to concentrate it. After decanting off excess ethanol, a lactose to ethanol ratio of 1:4 was achieved. This was added as a thick paste to the inner walls of PET vials into which micronised Formoterol fumarate had been added, then charged with propellant to prepare formulations as follows:

| | mg/ml | g/unit | mg/ml | g/unit | mg/ml | g/unit |
|---|---|---|---|---|---|---|
| Formo- | 0.132 | 0.0020 | 0.132 | 0.0017 | 0.132 | 0.0018 |
| terol | | | | | | |
| fumarate | | | | | | |
| Lactose | 2.640 | 0.0390 | 2.640 | 0.0340 | 2.640 | 0.0357 |
| mono- | | | | | | |
| hydrate | | | | | | |
| Ethanol | 12.180 | 0.1800 | 13.960 | 0.1800 | 13.317 | 0.1800 |
| HFA 134a | 1203.048 | 17.7790 | 0.000 | 0.0000 | 493.337 | 6.6684 |
| HFA 227 | 0.000 | 0.0000 | 1379.268 | 17.7843 | 822.228 | 11.1141 |

Each formulation was subjected to ultrasonic agitation in a water bath for one minute to ensure complete dispersion. The physical appearance was assessed visually and by using an optical measuring technique such as that described in the Proceedings of Drug Delivery to the Lung VI p. 10-13 (December 1995) printed by the Aerosol Society.

The formulations were compared with three formulations of the same composition, in which the lactose monohydrate had been air-jet milled to a micronised size range similar to that of the drug.

All of the formulations with lactose processed according to example 1 sedimented more slowly than all of those with air-jet milled lactose, and they were therefore more stable in this respect.

### Example 3

Further formulations were prepared in a similar manner to Example 2 using nanoparticulate lactose and micronised lactose but without drug as follows:

| | mg/ml | g/unit | mg/ml | g/unit | mg/ml | g/unit |
|---|---|---|---|---|---|---|
| Lactose | 2.772 | 0.0410 | 2.772 | 0.0357 | 2.772 | 0.0375 |
| mono- | | | | | | |
| hydrate | | | | | | |
| Ethanol | 12.180 | 0.1800 | 13.960 | 0.1800 | 13.317 | 0.1800 |
| HFA 134a | 1203.048 | 17.7790 | 0.000 | 0.0000 | 493.337 | 6.6684 |
| HFA 227 | 0.000 | 0.0000 | 1379.268 | 17.7843 | 822.228 | 11.1141 |

Similar results were obtained using the optical measuring technique to those obtained for the formulations of Example 2, samples containing nanoparticulate lactose sedimented more slowly than all those samples containing air-jet milled micronised lactose.

Further formulations were made on a scale of about 20 inhalers as follows:

| | mg/ml | g/unit | mg/ml | g/unit |
|---|---|---|---|---|
| Formoterol | 0.1320 | 0.0010 | 0.1320 | 0.0010 |
| Lactose monohydrate | 0.5280 | 0.0040 | 2.6400 | 0.0198 |
| Ethanol | 1.9975 | 0.0150 | 13.3165 | 0.1000 |
| HFA 134a | 493.3370 | 3.7047 | 493.337 | 3.7047 |
| HFA 227 | 822.2284 | 6.1745 | 822.228 | 6.1745 |

The formulations exhibited good visual stability.

### Example 5

Further formulations of Formoterol Fumarate were prepared on a small manufacturing scale (300 inhalers), in which a thick slurry of nanosized lactose prepared as in Example 2 but with a weight ratio of lactose:ethanol of 1:2.

The slurry was added to a stainless steel vessel. Oleic acid with any necessary additional ethanol was added. The mixture was homogenised for 5 minutes.

Propellants were added to a batching vessel. The slurry was added to the propellants. Some additional propellant was used to rinse the stainless steel vessel. Drug was then added to the batching vessel, and dispersed using a high shear mixer at 8000 rpm for one minute. The formulation was dispensed into canisters by cold-filling, then valves were crimped on.

| | mg/ml | g/unit | mg/ml | g/unit | mg/ml | g/unit |
|---|---|---|---|---|---|---|
| Formo- | 0.1320 | 0.0010 | 0.1320 | 0.0010 | 0.1320 | 0.0010 |
| terol | | | | | | |
| fuma- | | | | | | |
| rate | | | | | | |
| Lactose | 2.6400 | 0.0218 | 2.6400 | 0.0190 | 2.6400 | 0.0200 |
| mono- | | | | | | |
| hydrate | | | | | | |
| Oleic | 0.0606 | 0.0005 | 0.0695 | 0.0005 | 0.0661 | 0.0005 |
| Acid | | | | | | |
| Ethanol | 24.2285 | 0.2000 | 27.798 | 0.2000 | 26.4595 | 0.2000 |
| HFA | 1184.3653 | 9.7766 | 0.0000 | 0.0000 | 485.1290 | 3.6744 |
| 134a | | | | | | |
| HFA | 0.0000 | 0.0000 | 1359.2644 | 9.7796 | 808.5483 | 6.1241 |
| 227 | | | | | | |

Further formulations without lactose were made on a small manufacturing scale for comparison, as follows:

| | mg/ml | g/unit | mg/ml | g/unit | mg/ml | g/unit |
|---|---|---|---|---|---|---|
| Formo- | 0.1320 | 0.0011 | 0.132 | 0.0010 | 0.132 | 0.0010 |
| terol | | | | | | |
| fuma- | | | | | | |
| rate | | | | | | |
| Oleic | 0.0606 | 0.0005 | 0.0695 | 0.0005 | 0.0661 | 0.0005 |
| Acid | | | | | | |
| Ethanol | 24.2285 | 0.2000 | 27.7981 | 0.200 | 26.4595 | 0.2000 |
| HFA | 1187.0049 | 9.7984 | 0.0000 | 0.0000 | 486.119 | 3.6744 |
| 134a | | | | | | |
| HFA | 0.0000 | 0.0000 | 1361.9044 | 9.7986 | 810.1983 | 6.1241 |
| 227 | | | | | | |

Initial medication delivery data for the inhalers manufactured on a small scale showed that those formulations with nanosized lactose gave more accurate dosing of drug.

### Example 6

Micronised lactose monohydrate (100 g) was dispersed in anhydrous ethanol (600 g) for 1 minute using a Silverson high shear mixer. This dispersion was added to the product reservoir of an Avestin Emulsiflex C50 homogeniser, and passed through the homogeniser at 10,000 psi and again at 21,000 psi. Part of the resulting dispersion (411 g) was diluted with Anhydrous Ethanol to a weight of 645 g, to achieve a ratio of liquid:solid:10:1 and to allow further processing. This composition was passed through the homogeniser at 20,000 psi. A batch was used for particle analysis (see below) and another batch for formulation testing (see below).

### Example 7

Micronised lactose monohydrate (59.7 g) was dispersed in anhydrous ethanol (435.8 g) for 1 minute using a Silverson high shear mixer. This dispersion was added to the product reservoir of an Avestin Emulsiflex C50 homogenizer, and passed through the homogenizer at 10,000 psi and again at 20,000 psi. At the start of each passage, the dispersion was recirculated briefly to ensure that all product collected had passed through the homogenizer at the target pressure. The dispersion was then recirculated through the homogenizer continuously for 5 minutes at approximately 20,000 psi, and then sampled. Particle analysis was performed (see below).

### Example 8

Micronised lactose monohydrate (60.1 g) was dispersed in anhydrous ethanol (438.7 g) for 1 minute using a Silverson high shear mixer. This dispersion was added to the product reservoir of an Avestin Emulsiflex C50 homogenizer, and passed through the homogenizer at 10,000 psi and again twice at 20,000 psi. At the start of each passage, the dispersion was recirculated briefly to ensure that all product collected had passed through the homogenizer at the target pressure. The dispersion was then sampled. Particle analysis was performed (see below).

### Particle Size Analysis

For analysis of a Lactose/Ethanol slurry, a (0.5 ml) sample of the slurry, which was shaken for at least one minute to ensure homogeneity, was added to a solution of 0.05% Lecithin in Iso-octane (20 ml), and redispersed with mild ultrasonics for 1 minute.

For analysis of powdered Lactose (here double-micronised α-lactose monohydrate), a sample (500 mg) of the powder, which was previously shaken to ensure homogeneity, was added to a solution of 0.05% Lecithin in Iso-octane (20 ml), and dispersed with mild ultrasonics for 1 minute.

The resulting suspension was introduced dropwise into the presentation cell (a Hydro 2000 SM small sample presentation cell) of a Malvern Mastersizer 2000™ laser diffraction particle sizer until the obscuration was in the working range (between 10 and 12 with a red laser), and left to circulate for 1 minute to allow complete mixing and thermal equilibrium to be established. Ten readings were taken at 20 second intervals to establish that the particle size was stable. The General Purpose analysis model, as described in the Malvern Instruments Operators Guide, was used with refractive indices 1.533 (lactose), 1.392 (iso octane) and absorbance 0.001 (lactose). The results are based on the average calculated results of 10 readings taken in succession. The procedure was performed twice.

### Results of Particle Size Analysis by Malvern Mastersizer 2000

| | Lactose, | | | |
|---|---|---|---|---|
| | Double- | | | |
| | micronised by | | | |
| | fluid energy | Lactose, | Lactose, | Lactose, |
| | mill | Example 6 | Example 7 | Example 8 |
| Units | Microns | Microns | Microns | Microns |
| d(v, 0.1) | 2.103, 2.127 | 0.077, 0.077 | 0.086, 0.087 | 0.085, 0.084 |
| d(v, 0.5) median | 3.385, 3.590 | 0.193, 0.193 | 0.245, 0.250 | 0.250, 0.245 |
| d(v, 0.9) | 5.721, 6.338 | 1.092, 1.100 | 1.201, 1.294 | 1.568, 1.645 |
| D[4, 3] volume | 3.691, 3.966 | 0.421, 0.429 | 0.459, 0.487 | 0.590, 0.611 |
| weighted mean | | | | |
| Units | Percent | Percent | Percent | Percent |
| Vol under 0.05 micron | 0.000, 0.000 | 1.73, 1.72 | 1.22, 1.18 | 1.29, 1.32 |
| Vol under 0.10 micron | 0.000, 0.000 | 19.55, 19.50 | 14.48, 14.10 | 15.01, 15.34 |
| Vol under 0.20 micron | 0.000, 0.000 | 51.57, 51.49 | 41.73, 40.97 | 41.70, 42.37 |
| Vol under 0.50 micron | 0.000, 0.000 | 77.54, 77.50 | 72.10, 71.18 | 68.81, 68.57 |
| Vol under 1.0 micron | 0.000, 0.000 | 88.71, 88.62 | 86.42, 84.98 | 82.00, 81.22 |
| Vol under 2.0 micron | 7.49, 7.20 | 97.16, 97.00 | 97.70, 96.83 | 93.64, 92.98 |
| Vol under 5.0 micron | 82.69, 76.79 | 99.76, 99.66 | 100.00, 100.00 | 99.52, 99.38 |
| Vol under 10.0 micron | 100.00, 99.56 | 100.00, 100.00 | 100.00, 100.00 | 100.00, 100.00 |
| Vol under 20.0 micron | 100.00, 100.00 | 100.00, 100.00 | 100.00, 100.00 | 100.00, 100.00 |

### Formulation Testing

Lactose dispersion from Example 6 was used to formulate inhalers with the following formulations:

| | Example 9 | Example 10 | Example 11 | Example 12 |
|---|---|---|---|---|
| Formulation | % w/w | % w/w | % w/w | % w/w |
| Formoterol | 0.010 | 0.011 | 0.011 | 0.011 |
| Fumarate | | | | |
| Lactose | 0.200 | 0.218 | 0.109 | 0.327 |
| Oleic Acid | 0.005 | 0.005 | 0.005 | 0.005 |
| Ethanol | 2.000 | 2.000 | 2.000 | 2.000 |
| P134a | 36.670 | 97.766 | 97.875 | 97.657 |
| P227 | 61.116 | 0.000 | 0.000 | 0.000 |
| Total | 100.000 | 100.000 | 100.000 | 100.000 |

These were compared with equivalent formulations, in which the Lactose was prepared by double-micronising using a fluid energy mill, which are respectively designated formulations 9a-12a. (Mass median diameter, the average of the two values given above in the Table summarising the results of Particle Size Analysis, for Lactose (example 6) and double-micronised Lactose are 193 nm and 3.486 microns, respectively.)

It was found that the uniformity of delivered dose, as measured by relative standard deviation, was significantly better for the inhalers made using the Lactose prepared in example 6 than for those made from double-micronised Lactose.

Formulations, in which the ratio of Lactose to drug was 10:1 (Example 11) and 30:1 (Example 12) were compared with the formulation of example 10 (ratio 20:1). It was surprisingly found that the ratio of 20:1 gave superior uniformity of content than those with ratios 10:1 or 30:1.

| | Mean (n = 15) | RSD |
|---|---|---|
| Example | (mcg/actuation) | (%) |
| 12 | 5.2 | 7.1 |
| 10 | 4.9 | 6.6 |
| 11 | 5.8 | 8.2 |
| 12a | 5.7 | 15.1 |
| 10a | 5.5 | 12.7 |
| 11a | 5.2 | 14.2 |

Furthermore, the loss of dose from the metering tank of the valve, which occurs when the product is allowed to stand for a prolonged period in the valve-up orientation, was compared for formulations 9 and 10, and the respective formulations 9a and 10a prepared from double-micronised Lactose. A reduced loss of dose was found when the inhalers were prepared using Lactose prepared in example 6 compared with those made from double-micronised Lactose.

### Example 13

Micronised lactose monohydrate (100 g) was dispersed in Anhydrous Ethanol (840 g) using a Silverson high shear mixer. This dispersion was added to the product reservoir of an Avestin Emulsiflex C50 homogenizer, and recirculated for 20 minutes at 10,000 psi. The dispersion was then passed out of the homogenizer at 20,000 psi.

### Example 14

Micronised lactose monohydrate (1063 g) was dispersed in anhydrous ethanol (8929 g) for 10 minutes using a Silverson high shear mixer. The mixture was added to a 20 litre stainless steel vessel and re-circulated through an Avestin Emulsiflex C160 high pressure homogeniser for 50 minutes at a pressure setting of 10,000 psi. The resultant lactose/ethanol slurry was used to prepare the following lactose bulked suspension formulation of salbutamol sulphate:

| | Quantity (g) |
|---|---|
| Salbutamol sulphate (micronised) | 0.061 |
| Lactose/ethanol slurry | 0.571 (0.060 lactose/0.511 ethanol) |
| Ethanol | 1.94 |
| HFA 134a | 14.0 |

The formulation was prepared by weighing out the salbutamol sulphate into a clear plastic (PET) vial and then adding the lactose/ethanol slurry and ethanol. A non-metering valve was then crimped in place and the vial was sonicated in an ultrasonic water bath for one minute to disperse the solids. The HFA 134a was then injected into the vial to complete the formulation. A second vial (unbulked formulation) was prepared in which the lactose/ethanol slurry was omitted and replaced by a further quantity of ethanol (0.51 g).

The lactose bulked and the unbulked formulations of salbutamol sulphate were then visually compared and it was noted that the sedimentation rate was much slower for the bulked formulation. Floc heights were measured two minutes after shaking both vials. After two minutes, the floc height of the unbulked formulation filled 33% of the formulation volume, whereas the floc height of the bulked formulation filled 95% of the formulation volume. This observation indicates that by incorporation of sub-micron lactose into the formulation, a more dispersed and uniform suspension has been achieved.

### Example 15

DL-alanine (10 g, used as supplied by Fisher Chemicals, Loughborough UK) was dispersed in anhydrous ethanol (200 g) using a Silversen high shear mixer set at 10,000 RPM for 1 minute. The resultant dispersion was poured into the product vessel of a M110EH Microfluidizer. The Microfluidiser was fitted with an auxiliary process module of 100 micron channel diameter and a G10Z diamond interaction chamber of 87 micron channel diameter. The G10Z chamber was fitted downstream from the auxiliary process module. The dispersion was processed at 25,000 psi for 120 minutes.

A particle size analysis measurement was performed using a Malvern Mastersizer 2000™ laser diffraction particle sizer in an analogous manner to that described above for lactose, with the exception that a refractive index of 1.55 was employed for DL-alanine.

### Results of Particle Size Analysis by Malvern Mastersizer 2000

| | DL-Alanine |
|---|---|
| Units | Microns |
| d(v, 0.1) | 0.077 |
| d(v, 0.5) median | 0.190 |
| d(v, 0.9) | 0.600 |
| D[4, 3] volume | 0.283 |
| weighted mean | |
| Units | Percent |
| vol under 0.05 micron | 1.68 |
| vol under 0.10 micron | 19.13 |
| vol under 0.20 micron | 52.45 |
| vol under 0.50 micron | 86.56 |
| vol under 1.0 micron | 96.25 |
| vol under 2.0 micron | 99.98 |
| vol under 5.0 micron | 100.00 |
| vol under 10.0 micron | 100.00 |
| vol under 20.0 micron | 100.00 |

### Example 16

Sucrose (20 g, used as supplied by British Sugar under the designation “Silk Sugar” (i.e. a fine grain icing sugar)) was dispersed in anhydrous ethanol (400 g) using a Silverson high shear mixer set at 10,000 RPM for 1 minute. The resultant dispersion was poured into the product vessel of a M110EH Microfluidizer. The Microfluidizer was fitted with an auxiliary process module of 100 micron channel diameter and a G10Z diamond interaction chamber of 87 micron channel diameter. The G10Z chamber was fitted downstream from the auxiliary process module. The dispersion was processed at 25,000 psi for 60 minutes. A sample of the processed particles was examined by SEM, observed particle size was found in the range of 200 to 800 nanometers.

### Example 17

A formulation of salbutamol sulphate was prepared using DL-alanine/ethanol slurry from Example 15 as follows.

| | Quantity (g) |
|---|---|
| Salbutamol sulphate (micronised) | 0.061 |
| DL-alanine/ethanol slurry | 2.57 (0.12 DL-alanine/2.45 ethanol) |
| P134a | 14.0 |

The formulation was prepared by weighing out the salbutamol sulphate into a clear plastic PET vial and then adding the DL-alanine/ethanol slurry. A non-metering valve was then crimped in place and the vial was sonicated in an ultrasonic water bath for 1 minute to disperse the solids. The P134a was then injected into the vial to complete the formulation. A second vial (unbulked formulation) was then prepared in which the DL-alanine/ethanol slurry was replaced by ethanol (2.45 g).

The DL-alanine bulked and the unbulked formulations of salbutamol sulphate were visually compared and it was noted that the sedimentation rate was much slower for the bulked formulation. Floc heights were measured two minutes after shaking both vials. After two minutes, the floc height of the unbulked formulation filled 34% of the formulation volume, whereas floc height of the bulked formulation filled 99% of the formulation volume. This observation indicates that by incorporation of sub-micron DL-alanine into the formulation, a more uniform and dispersed suspension had been achieved.

### Example 18

A formulation of salbutamol sulphate was prepared using sucrose/ethanol slurry from Example 16 as follows:

| | Quantity (g) |
|---|---|
| Salbutamol sulphate (micronised) | 0.061 |
| sucrose/ethanol slurry | 2.57 (0.12 sucrose/2.45 ethanol) |
| P134a | 14.0 |

The formulation was prepared by weighing out the salbutamol sulphate into a clear plastic PET vial and then adding the sucrose/ethanol slurry. A non-metering valve was then crimped in place and the vial was sonicated in an ultrasonic water bath for 1 minute to disperse the solids. The P134a was then injected into the vial to complete the formulation.

The sucrose bulked and the unbulked formulations of salbutamol sulphate were compared visually and it was noted that the sedimentation rate was much slower for the bulked formulation. Floc heights were measured two minutes after shaking both vials. After two minutes, the floc height of the unbulked formulation filled 34% of the formulation volume, whereas the floc height of the sucrose bulked formulation filled 80% of the formulation volume. This observation indicates that by incorporation of sub-micron sucrose into the formulation, a more uniform and dispersed suspension had been achieved.

1-38. (canceled)

39. A pharmaceutical aerosol formulation comprising particles of drug dispersed in a propellant and a bulking agent having a mass median diameter of less than one micron.

40. A pharmaceutical aerosol formulation as claimed in claim 39 in which the bulking agent is selected from ascorbic acid, saccharides, polysaccharides, amino acids, organic and inorganic salts, urea and propyliodone.

41. A pharmaceutical aerosol formulation as claimed in claim 40 in which the bulking agent is selected from lactose, DL-alanine, glucose, D(+)trehalose dihydrate, sucrose, maltose, D(+)raffinose pentahydrate, sodium saccharin, starches, modified celluloses, dextrins, dextrans, glycine, sodium chloride, calcium carbonate, sodium tartrate and calcium lactate.

42. A pharmaceutical aerosol formulation as claimed in claim 39 in which the bulking agent is lactose.

43. A pharmaceutical aerosol formulation as claimed in claim 39 in which the bulking agent has a mass median diameter of not more than 300 nm.

44. A pharmaceutical aerosol formulation as claimed in claim 39 in which the weight ratio of drug:auxiliary agent is in the range 1:3 to 1:100.

45. A pharmaceutical aerosol formulation as claimed in claim 44 in which the weight ratio of drug:bulking agent is in the range 1:5 to 1:40.

46. A pharmaceutical aerosol formulation as claimed in claim 39 in which the drug is micronized.

47. A pharmaceutical aerosol formulation as claimed in claim 39 in which the drug has a mass median diameter of less than one micron.

48. A pharmaceutical aerosol formulation as claimed in claim 39 in which the drug is selected from the group consisting of formoterol, salmeterol, fluticasone propionate, procaterol and ipratropium and salts thereof.

49. A pharmaceutical aerosol formulation as claimed in claim 48 in which the drug is formoterol or a salt thereof.

50. A pharmaceutical aerosol formulation as claimed in claim 39 in which the propellant is a hydrofluoroalkane.

51. A pharmaceutical aerosol formulation as claimed in claim 50 in which the propellant is selected from HFA 134a, HFA 227 and mixtures thereof.

52. A pharmaceutical aerosol formulation as claimed in claim 39 which additionally comprises a surfactant.

53. A pharmaceutical aerosol formulation as claimed in claim 52 in which the surfactant is selected from sorbitan trioleate, oleic acid and oligolactic acid derivatives.

54. A pharmaceutical aerosol formulation as claimed in claim 39 which additionally comprises ethanol.

55. A pharmaceutical aerosol formulation as claimed in claim 54 in which the ethanol comprises from 0.1 to 5% by weight of the formulation.

56. A pharmaceutical aerosol formulation as claimed in claim 55 in which the ethanol comprises from about 1 to about 2% by weight of the formulation.

57. A process for the preparation of an aerosol formulation comprising a suspension of particles in propellant which process comprises forming a slurry of bulking agent with a component of the aerosol formulation, subjecting the slurry to high pressure homogenization to reduce the particle size of the bulking agent, and thereafter mixing the resulting slurry with other components of the aerosol formulation.

58. A process as claimed in claim 57 in which the slurry of bulking agent has a liquid:solid weight ratio of from 10:1 to 100:1.

59. A process as claimed in claim 58 in which the slurry of bulking agent has a liquid:solid weight ratio of from 20:1 to 40:1.

60. A process as claimed in claim 57 comprising the step of adjusting the liquid:solid ratio of the slurry after homogenisation before mixing with other components of the aerosol formulation.

61. A process as claimed in claim 57 in which the bulking agent is reduced to particles having a mass median diameter of not more than one micron.
